(19) United States
(12) Patent Application Publication
(10) Pub. No.: US 2026/0301222 A1
(43) Pub. Date: Oct. 1, 2026

(54) METHOD FOR CREATING A SPATIAL GRAPHICAL REPRESENTATION OF AN ORGAN OF A BODY

(71) Applicant: AIMITARI MEDTECH PTE. LTD., Singapore (SG)

(72) Inventors: Boon Huat LIM, Singapore (SG); Ravintharan S/O THARMALINGAM, Singapore (SG); Sebastian Johan ZILLIACUS, Singapore (SG); Brian James TRACEY, Singapore (SG); Rajashekar DURAI, Singapore (SG)

(21) Appl. No.: 19/479,369

(22) PCT Filed: Apr. 28, 2023
(86) PCT No.: PCT/SG2023/050293

§ 371 Date: Oct. 28, 2025

(51) Int. Cl. G06T 7/73; A61B 8/00; G06T 17/00; G16H 40/63

(52) U.S. Cl. CPC G06T 7/75; A61B 8/4254; A61B 8/461; G06T 17/00; G16H 40/63; G06T 2207/10016; G06T 2207/10132; G06T 2207/30004; G06T 2210/41; G06T 2210/56

(57) ABSTRACT

A method for creating a spatial graphical representation 4 of an organ of a body 5 from a multitude of 2-dimensional ultrasound images 8, the method comprising the following steps: —providing a medical ultrasound transducer system 1 with an ultrasound transducer unit 6 and with a display unit 7 for displaying a digital video signal as a 2D Ultrasound image 8 that represents what the ultrasound transducer unit 6 captures during the operation of the medical ultrasound transducer system 1.

The present application relates generally to methods for creating graphical representations of internal body 5 organs, and more specifically to a novel method for generating a spatial graphical representation 4 of an organ within a body 5 using imaging and computer processing techniques.

This application addresses the problem of creating a three-dimensional representation of an organ or other body 5 tissue from two-dimensional ultrasound images 8 by providing a method for accurately measuring the spatial position and orientation of the ultrasound transducer unit 6 and using this information to create a 3D-model of the body 5 structure. This method improves the accuracy and reliability of medical diagnoses and treatments for conditions affecting internal organs and tissues 3.

There is disclosed a method for creating a spatial graphical representation 4 of an organ of a body 5 from a multitude of 2-dimensional ultrasound images 8, the method comprising the following steps:
providing a medical ultrasound transducer system 1 with an ultrasound transducer unit 6 and with a display unit 7 for displaying a digital video signal as a 2D Ultrasound image 8 that represents what the ultrasound transducer unit 6 captures during the operation of the medical ultrasound transducer system 1,
providing a spatial attitude measurement unit 9 that is attached to the medical ultrasound transducer 11 unit, wherein the attitude measurement unit 9 comprises at least one linear accelerometer 10 and/or at least one rotational gyroscope 10 for each one of at least three spatial directions x, y, z, α, β, γ, wherein the at least one linear accelerometer 10 and/or the at least one rotational gyroscope 10 are provided in MEMS technology,
providing a measurement computer system 14 that is electrically connected with the display unit 7 and the attitude measurement unit 9, and that is provided for receiving and storing
a plurality of digital video signals, and other common meanings of “signal”) and
a plurality of digital attitude measurement signals 19 from the attitude measurement unit 9,
a display unit 7, instead of integrating the Smart Sensor 9 Technology into the Medical Ultrasound transducer system 1, and works with the video data) in an initialization phase:
moving the medical ultrasound transducer 11 to an initial attitude, initializing the spatial position and/or orientation measurement unit, and recording an initial video signal together with an initial attitude in a recording phase:
moving the medical ultrasound transducer 11 to a series of consecutive delta attitudes, a preceding delta attitude being different from a following delta attitude,
recording a series of delta video signals together with an associated series of delta attitude measurement signals,, in an evaluation phase:
inspecting the recorded initial video signal and the recorded series of delta video signals for image points that are characteristic of a surface of a pre-determined bodystructure, can be organs or other body 5 tissues 3, such as bones, tendons, muscles, ligaments, etc. and can also be a characteristic surface of a changed surface structure, such as a scar, or an interrupted or missing area, such as a wound),
determining the absolute positions of the characteristic surface image points from the associated delta attitude measurement signals,
providing a 3D-model of the pre-determined bodystructure by outputting the characteristic surface image points together with their absolute positions.

The use of Smart Sensor 9 Technology allows for accurate and precise spatial attitude measurements, resulting in a more accurate 3D-model of the body 5 structure.

The recording of a series of delta video signals together with their associated delta attitude measurement signals allows for the determination of absolute positions of characteristic surface image points, resulting in a more accurate 3D-measurement model.

The ability to enter labeling data that is characteristic for body structures 13 to recorded delta video signals allows for easy identification and tracking of specific body structures 13.

In a development, the recording phase comprises recording a series of digital camera signals together with the associated series of delta attitude measurement signals.

The use of a series of digital camera signals together with their associated delta attitude measurement signals in the recording phase provides a more comprehensive and detailed view of the body 5 structure being imaged.

The application of comparing the 3D-measurement model with pre-recorded 3D-control models allows for quality control and accuracy of the resulting 3D-model.

The ability to record additional delta video signals provides more data for accuracy in the creation of the 3D-model.

In a development, the application provides A method comprising the step of reading digital information that is obtained from comparing the 3D-measurement model with at least one pre-recorded 3D-control model from a display and providing recording additional delta video signals.

The use of Smart Sensor 9 Technology results in accurate and precise spatial attitude measurements, leading to a more accurate 3D-model of the body 5 structure.

The ability to read digital information obtained from comparing the 3D-measurement model with pre-recorded 3D-control models allows for quality control and accuracy of the resulting 3D-model.

The possibility of entering labeling data that is characteristic for body structures 13 to recorded delta video signals allows for easy identification and tracking of specific body structures 13.

In a development, the application provides A method according to comprising the step of entering labeling data that is characteristic for body structures 13 to recorded delta video signals.

The use of Smart Sensor 9 Technology leads to accurate and precise spatial attitude measurements, providing a more accurate 3D-model of the body 5 structure.

The ability to read digital information obtained from comparing the 3D-measurement model with pre-recorded 3D-control models allows for quality control and accuracy of the resulting 3D-model.

The possibility of entering labeling data that is characteristic for body structures 13 to recorded delta video signals allows for easy identification and tracking of specific body structures 13.

In a further development with measurement computer system 14 for providing a spatial graphical representation 4 of an organ of a body 5 from a multitude of 2-dimensional ultrasound images 8, the measurement computer system 14 comprises:
a video input connector 18 for receiving a digital video signal that a medical ultrasound transducer system 1 sends to a display unit 7, the video signal comprises a 2D Ultrasound picture 8 that represents what the ultrasound transducer unit 6 captures during the operation of the medical ultrasound transducer system 1,
an attitude input connector 17 for receiving a digital attitude signal from a spatial attitude measurement unit 9 that is attachable to a medical ultrasound transducer 11, comprises at least one linear accelerometer 10 and at least one rotational gyroscope 10 for each one of at least three spatial directions, and the at least one linear accelerometer 10 and the at least one rotational gyroscope 10 are provided in MEMS technology)
wherein the measurement computer system 14 is provided for receiving and storing a plurality of digital video signals, and other common meanings of “signal”) and
a plurality of digital attitude measurement signals 19),
display unit 7, instead of integrating the Smart Sensor 9 Technology into the Medical Ultrasound transducer system 1, and works with the video data) wherein the measurement computer system 14 is further provided for:
recording a series of delta video signals together with an associated series of delta attitude measurement signals, and for
inspecting the recorded initial video signal and the recorded series of delta initial video signals for image points that are characteristic of a surface of a pre-determined bodystructure, can be organs or other body 5 tissues 3, such as bones, tendons, muscles, ligaments, etc and can also be a characteristic surface of a changed surface structure, such as a scar, or an interrupted or missing area, such as a wound), and for
determining the absolute positions of the characteristic surface image points S from the associated delta attitude measurement signals, and for
providing a 3D-measurement model of the pre-determined bodystructure by outputting the characteristic surface image points together with their absolute positions.

The use of Smart Sensor 9 Technology provides accurate and precise spatial attitude measurements, leading to a more accurate 3D-model of the body 5 structure.

The recording of a series of delta video signals together with their associated delta attitude measurement signals allows for the determination of absolute positions of characteristic surface image points, resulting in a more accurate 3D-measurement model.

The ability to enter labeling data that is characteristic for body structures 13 to recorded delta video signals allows for easy identification and tracking of specific body structures 13.

In a development, the application provides a measurement computer system 14 with a video input connector 18 for receiving a digital camera signal from the spatial attitude measurement unit 9.

The measurement computer system 14 with a video input connector 18 enables the use of a digital camera signal for spatial attitude measurement, which provides higher resolution and accuracy than traditional analog signals.

The presence of a display output connector allows for real-time visualization of the spatial graphical representation 4 of an organ, providing immediate feedback to medical professionals.

The use of MEMS technology in the linear accelerometer and/or rotational gyroscope provides a compact and reliable solution for measuring spatial attitude.

In a development, the application provides A measurement computer system 14 with a display output connector for sending digital information that is obtained from comparing the 3D-measurement model with at least one pre-recorded 3D-control model.

The ability to compare the 3D-measurement model with pre-recorded 3D-control models allows for efficient and reliable quality control.

The display output connector enables the visualization of the comparison between the 3D models, facilitating the analysis of differences and potential errors.

The digital information obtained from the comparison can be easily stored, shared, and analyzed, providing valuable insights for medical research and clinical practice.

In a development, spatial attitude measurement unit 9 for providing a plurality of digital attitude measurement signals 19 to a measurement computer system 14 for providing a spatial graphical representation 4 of an organ of a body 5 from a multitude of 2-dimensional ultrasound images 8, the spatial attitude measurement unit 9 comprises an attitude output connector for sending a digital attitude signal and a digital camera module for supplying a digital camera signal, and the spatial attitude measurement unit 9 is attachable to a medical ultrasound transducer 11, wherein the attitude measurement unit 9 comprises at least one linear accelerometer 10 and/or at least one rotational gyroscope 10 for each one of at least three spatial directions, wherein the at least one linear accelerometer 10 and the at least one rotational gyroscope 10 are provided in MEMS technology.

The use of multiple linear accelerometers and/or rotational gyroscopes for each spatial direction enables a more comprehensive and accurate measurement of spatial attitude.

The attachment of the spatial attitude measurement unit 9 to a medical ultrasound transducer 11 allows for seamless integration into existing medical equipment and workflows.

The provision of multiple digital attitude signals and a digital camera signal allows for the creation of a high-quality spatial graphical representation 4 of an organ, enabling more detailed analysis and diagnosis.

The term “medical ultrasound transducer system 1” is used herein to refer to a system that includes at least one ultrasound transducer unit 6 and at least one other ultrasound transducer.

The term “spatial graphical representation 4” may refer to a 2d-representation of an organ of a body 5 from a multitude of 2-dimensional ultrasound images 8.

An “ultrasound transducer head 6” (in the sense of a medical ultrasound transducer 11 unit, in which at least one ultrasound transducer) designates: attached.

A “transducer head 6” designates: a unit of an ultrasound transducer that comprises the ultrasound transducer unit 6.

A “STANDARD BRAND AGNOSTIC ULTRASOUND TRANSDUCERS 6” designates: medical ultrasound transducers that are used by the medical ultrasound device manufacturer to provide the medical ultrasound device with the ultrasound images 8 that are displayed in the medical ultrasound device.

The term “ultrasound transducer unit 6” is used herein to refer to a unit that provides ultrasound signals.

The term “display unit 7” may refer to a display unit 7, wherein the digital video signals are recorded and/or stored.

The term “ultrasound image 8” may refer to a 2d-spatial representation of an organ of a body 5 from a multitude of 2-dimensional ultrasound images 8, wherein the term “signal” may refer to a signal that represents what the medical ultrasound transducer system 1 captures during operation.

The term “smart sensor 9” is used herein to refer to a device that can detect position data, such as an accelerometer or a rotational gyroscope. Attached to a medical ultrasound transducer 11, wherein the attitude measurement unit 9 may be a linear or rotational gyroscope.

A “gyroscope sensor 10” designates: any sensor that indicates an orientation of a body 5 relative to gravity.

The term “at least one linear accelerometer 10 and/or the at least one rotational gyroscope 10” is used herein to refer to one or both linear accelerometers and rotational gyroscopes.

The term “at least one rotational gyroscope 10” may refer to a linear accelerometer and/or a rotational gyroscope for each of at least three spatial directions.

A “pre-determined body” designates: a specific part of the body 5, such as for example the heart, the lungs, the liver, the kidneys, the prostate, etc. The term “body structures 13” may refer to organs or other body 5 tissues 3, such as bones, tendons, muscles, ligaments, etc.

A “smart pad 14” can designate a device that has a camera.

The term “measuring computer system” refers to a device that is capable of recording and/or processing data, such as video signals, audio signals, etc.

The term “display unit 7” may also refer to a display unit 7 that displays information on a display.

The term “display input connector 16” may refer to a connector that is connected to the display unit 7, wherein the signal sent by the transducer can be read by a digital camera.

The term “attitude input connector 17” may refer to a connector that can be used for receiving a signal. The term “video input connector 18” may refer to a connector that can be used for receiving a video signal for a display unit 7.

The term “digital attitude measurement signals 19” may refer to a series of delta attitude measurement signals, and other common meanings of “signal” may refer to a series of delta attitude measurement signals.

A hd camera 111 designates: the fact that these cameras are capable of generating video signals with a resolution of at least 720×480 pixels.

The term “linear accelerometer” may refer to a linear accelerometer or a rotational gyroscope. The term “rotational gyroscope” may refer to a linear accelerometer and/or a rotational gyroscope for each one of at least three spatial directions, such as x, y, z, etc.

The term “display unit 7” may refer to a display unit 7, wherein the digital video signals are recorded and/or stored.

Attached to a medical ultrasound transducer 11, wherein the attitude measurement unit 9 may be a linear or rotational gyroscope.

The term “measuring computer system” refers to a device that is capable of recording and/or processing data, such as video signals, audio signals, etc.

The term “medical ultrasound transducer system 1” is used herein to refer to a system that includes at least one ultrasound transducer unit 6 and/or at least one other ultrasound transducer. Attached to a medical ultrasound transducer 11, wherein the attitude measurement unit 9 may be a linear or rotational gyroscope.

The term “medical ultrasound transducer 11” is used herein to refer to a medical ultrasound transducer system 1 that includes an ultrasound transducer unit 6, wherein the term “medical ultrasound transducer 11” may also refer to a medical ultrasound transducer 11 unit.

The term “pre-determined bodystructure” may refer to organs or other body 5 tissues 3, such as bones, tendons, muscles, ligaments, etc.

A “2-dimensional ultrasound images 8” (in the sense of a series of 2-dimensional ultrasound images 8, a 2-dimensional ultrasound image 8 being the image that a medical ultrasound transducer system 1 produces of a body 5 organ that is scanned by the medical ultrasound transducer system 1, and) designates: a representation of the scanned organ on a 2-dimensional image plane—the

The term “delta video signals” may refer to a series of consecutively moving delta attitudes.

The term “associated series” may refer to a series of delta attitude measurement signals that are recorded together with an associated video signal.

The term “spatial graphical representation 4” may refer to a 2d-representation of an organ of a body 5 from a multitude of 2-dimensional ultrasound images 8.

A “body 5” (in the sense of organic structure, physical structure, dead body 5, torso, trunk, consistency, consistence, eubstance, soundbox) designates one or more of: the entire structure of an organism (an animal, plant, or human being); a group of persons associated by some common tie or occupation and regarded as an entity; a natural object consisting of a dead animal or person; an individual 3-dimensional object that has mass and that is distinguishable from other objects; the body 5 excluding the head and neck and limbs; a collection of particulars considered as a system; the property of holding together and retaining its shape; the central message of a communication; the main mass of a thing; a resonating chamber in a musical instrument (as the body 5 of a violin); the external structure of a vehicle.

The term “ultrasound transducer unit 6” is used herein to refer to a unit that provides ultrasound signals.

The term “spatial attitude” may refer to a position of the transducer in relation to an orientation of the body 5, such as x, y, z, etc.

A “MEMS technology” designates: a method and a technology that allows the formation of very small structures and devices by the application of surface microfabrication techniques (MEMS) such as lithography, etching, sputtering and/or other similar techniques, and which are used for microtechnology or micro-electromechanical

The term “characteristic surface image” may refer to a 2d-spatial picture 8 that is obtained from the position of the transducer in relation to the body 5 structure.

Providing a spatial attitude measurement unit that is tightly attached to the medical ultrasound transducer unit, wherein the attitude measurement unit comprises at least one linear accelerometer and/or at least one rotational gyroscope for each one of at least three different spatial directions x, y, z, α, β, γ, wherein the at least one linear accelerometer and/or the at least one rotational gyroscope are provided in MEMS technology. It is not necessary to use MEMS technology, any other conventional technology can be used as well. And these can also be only linear accelerometers or only rotational gyroscopes, instead of using combinations of linear accelerometers and rotational gyroscopes.

Providing a measurement computer system that is electrically connected with the display unit and the attitude measurement unit, and that is provided for receiving and storing allows for harvesting a plurality of digital video signals. The term “signal” represents the signal line, the electric signal on that signal line, over time, also the individual digital data sets that represent the pictures that are displayed on the display unit 7 (7), and other common meanings of “signal”.

A plurality of digital attitude measurement signals from the attitude measurement unit are harvested and the application taps and stores the video signals that are sent to the Medical Ultrasound transducer system display unit. A Y-splitter can be provided in that video cable. That is much more convenient than integrating the Smart Sensor Technology into the Medical Ultrasound transducer system, and it works with the video data alone.

In an initialization phase, the following is done:
moving the medical ultrasound transducer to an initial attitude, initializing the spatial position and/or orientation measurement unit, and recording an initial video signal together with an initial attitude in a recording phase:
moving the medical ultrasound transducer to a series of consecutive delta attitudes, each a preceding delta attitude being different from a following delta attitude,
recording a series of delta video signals together with an associated series of delta attitude measurement signals, meaning that there are unique pairs of associated delta video signals and delta attitude measurement signals.

In an evaluation phase, the following is done:
inspecting the recorded initial video signal and the recorded series of delta video signals for image points that are characteristic of a surface of a pre-determined body structure. That can be an animal or human body, it can be organs or other body tissues, such as bones, tendons, muscles, ligaments, etc. and can also be a characteristic surface of a changed surface structure, such as a scar, or an interrupted or missing area, such as a wound,
determining the absolute positions of the characteristic surface image points from the associated delta attitude measurement signals,
providing a 3D-model of the pre-determined bodystructure by outputting the characteristic surface image points together with their absolute positions.

The recording phase comprises recording a series of digital camera signals together with the associated series of delta attitude measurement signals, meaning that there are unique pairs of associated camera signals and delta attitude measurement signals.

The step of reading digital information that is obtained from comparing the 3D-measurement model with at least one pre-recorded 3D-control model from a display and providing recording additional delta video signals, such as percentage of the already scanned area of the pre-determined body structure.

A measurement computer system for providing a spatial graphical representation of an organ of a living body from a multitude of 2-dimensional ultrasound images, the measurement computer system comprising:
a video input connector for receiving a digital video signal that a medical ultrasound transducer system sends to a display unit, the video signal comprising a 2D Ultrasound picture that represents what the ultrasound transducer unit captures during the operation of the medical ultrasound transducer system,
an attitude input connector for receiving a digital attitude signal from a spatial attitude measurement unit that is tightly attachable to a medical ultrasound transducer, wherein the attitude measurement unit comprises at least one linear accelerometer and at least one rotational gyroscope for each one of at least three different spatial directions, wherein the at least one linear accelerometer and the at least one rotational gyroscope can be provided in MEMS technology or in conventional technology

The measurement computer system is provided for receiving and storing
a plurality of digital video signals, wherein the term “signal” represents the signal line, the electric signal on that signal line, over time, also the individual digital data sets that represent the pictures that are displayed on the display unit, and other common meanings of “signal”, and
a plurality of digital attitude measurement signals from the attitude measurement unit,

The application taps and stores the video signals that are sent to the Medical Ultrasound transducer system display unit, instead of integrating the Smart Sensor Technology into the Medical Ultrasound transducer system, and works with the video data, wherein the measurement computer system is further provided for:
recording a series of delta video signals together with an associated series of delta attitude measurement signals, meaning that there are unique pairs of associated delta video signals and delta attitude measurement signals, and for
inspecting the recorded initial video signal and the recorded series of delta initial video signals for image points that are characteristic of a surface of a pre-determined body structure, in the form of an animal or human body, can be organs or other body tissues, such as bones, tendons, muscles, ligaments, etc. and can also be a characteristic surface of a changed surface structure, such as a scar, or an interrupted or missing area, such as a wound, and for
determining the absolute positions of the characteristic surface image points S from the associated delta attitude measurement signals, and for
providing a 3D-measurement model of the pre-determined body structure by outputting the characteristic surface image points together with their absolute positions.

A measurement computer system is provided with a display output connector for sending digital information that is obtained from comparing the 3D-measurement model with at least one pre-recorded 3D-control model, such as percentage of the already scanned area of the pre-determined body structure.

A spatial attitude measurement unit for providing a plurality of digital attitude measurement signals to a measurement computer system for providing a spatial graphical representation of an organ of a living or dead body from a multitude of 2-dimensional ultrasound images, the spatial attitude measurement unit comprising an attitude output connector for sending a digital attitude signal and a digital camera module for supplying a digital camera signal, wherein the spatial attitude measurement unit is tightly attachable to a medical ultrasound transducer, wherein the attitude measurement unit comprises at least one linear accelerometer and/or at least one rotational gyroscope for each one of at least three different spatial directions, wherein the at least one linear accelerometer and the at least one rotational gyroscope can be provided in MEMS technology or conventional technology. It can also be only linear accelerometers or only rotational gyroscopes applied.

The present disclosure is illustrated by way of example and not limited in the accompanying figures in which like reference numerals indicate similar elements. Embodiments of the application will now be described with reference to the attached drawings

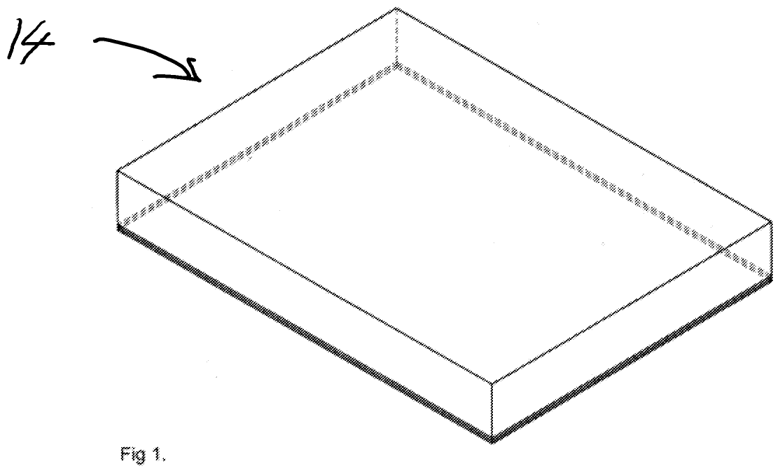

FIG. 1 shows a spatial view of a Smart Pad 14 according to the application,

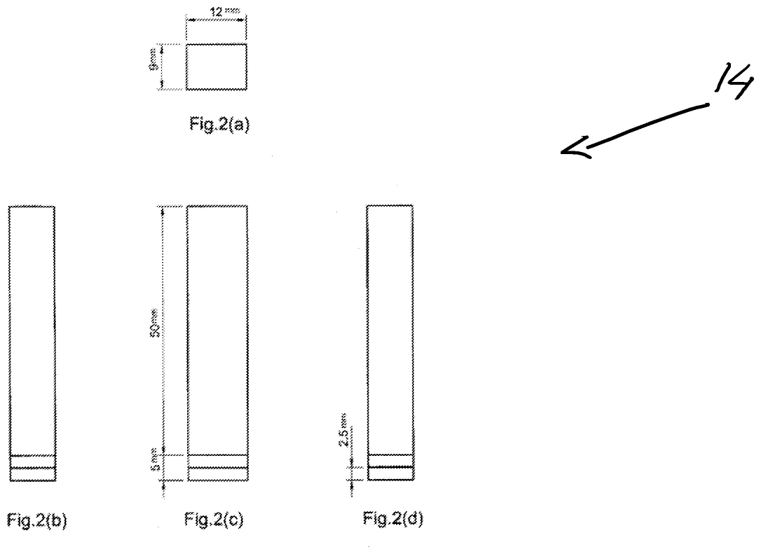

FIG. 2 shows a cross-sectional view of the Smart Pad 14 with two layers of embedded liquid metal filaments,

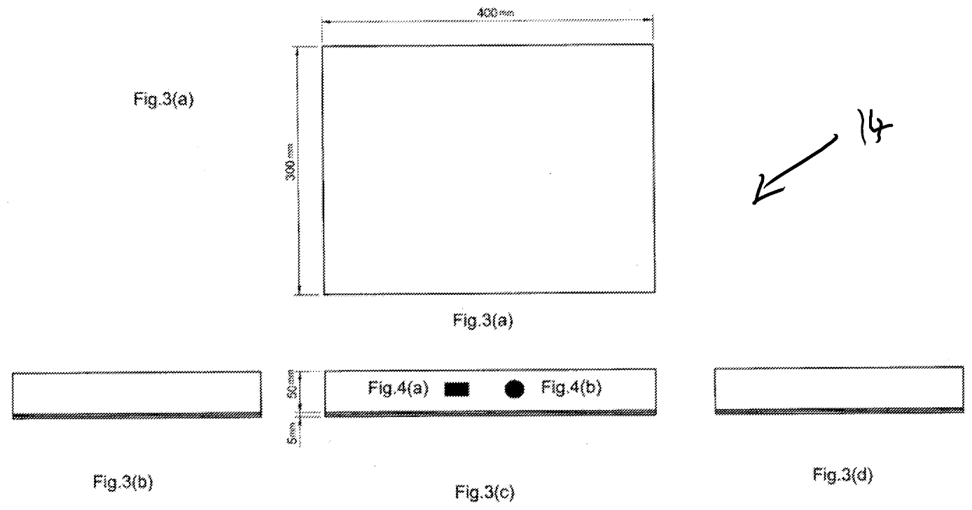

FIG. 3 shows another view of the Smart Pad 14 according to the application,

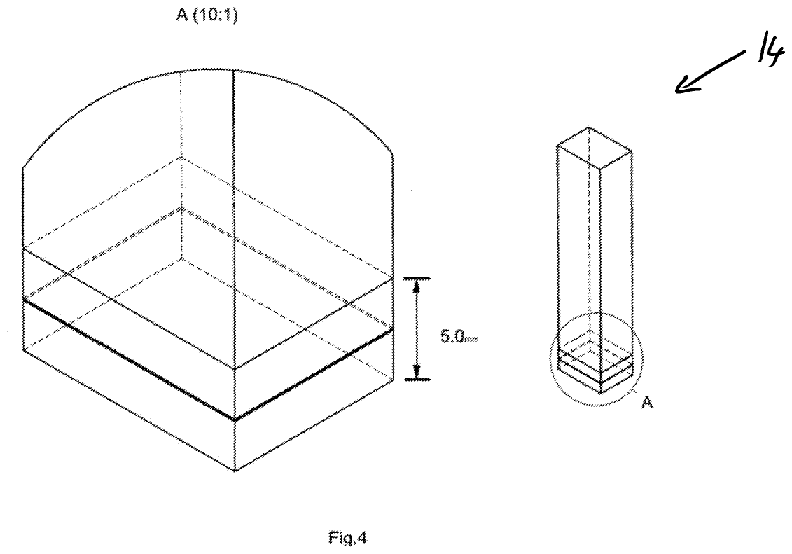

FIG. 4 shows a spatial view of FIG. 2,

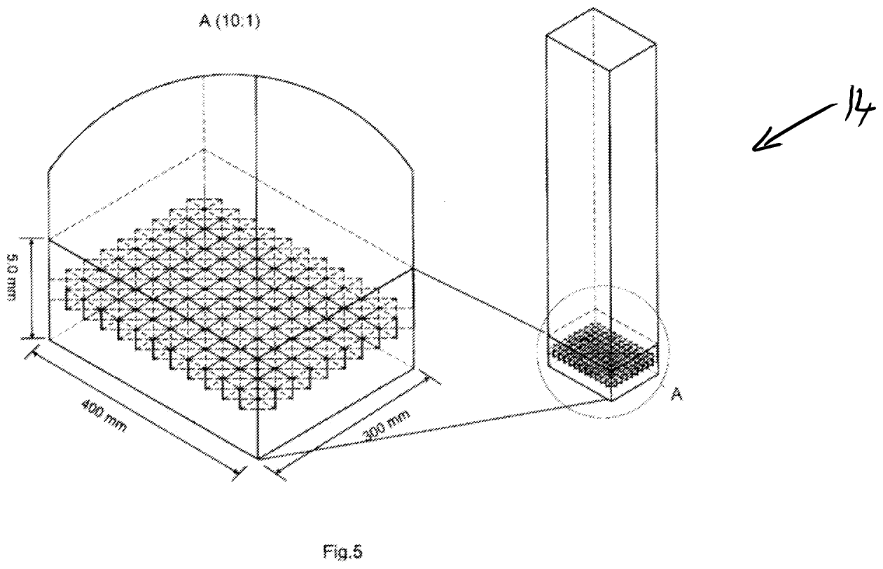

FIG. 5 shows the spatial view of FIG. 4 with the pattern matrix of the embedded liquid metal filaments,

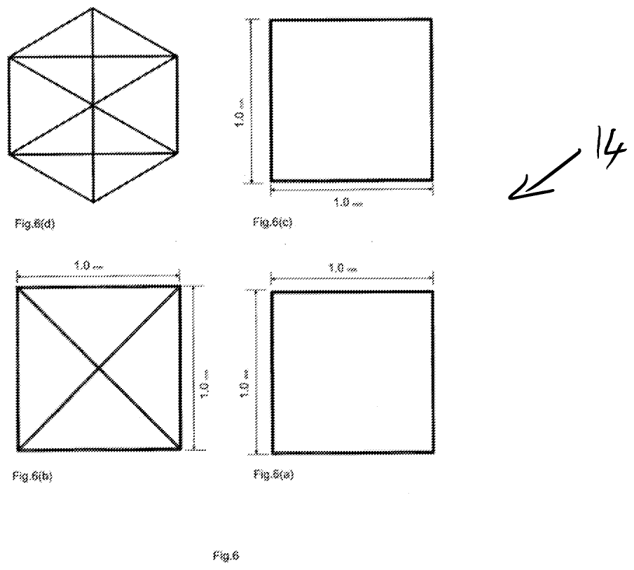

FIG. 6 shows the spacing and pattern of the embedded liquid metal filaments,

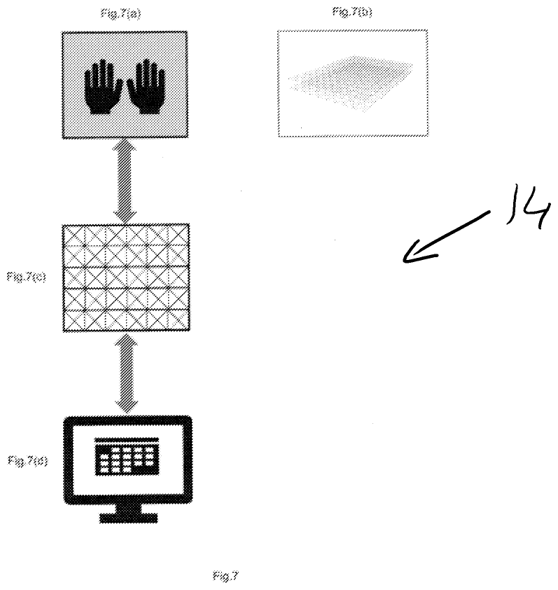

FIG. 7 shows systems overview of the Smart Pad 14 of the application,

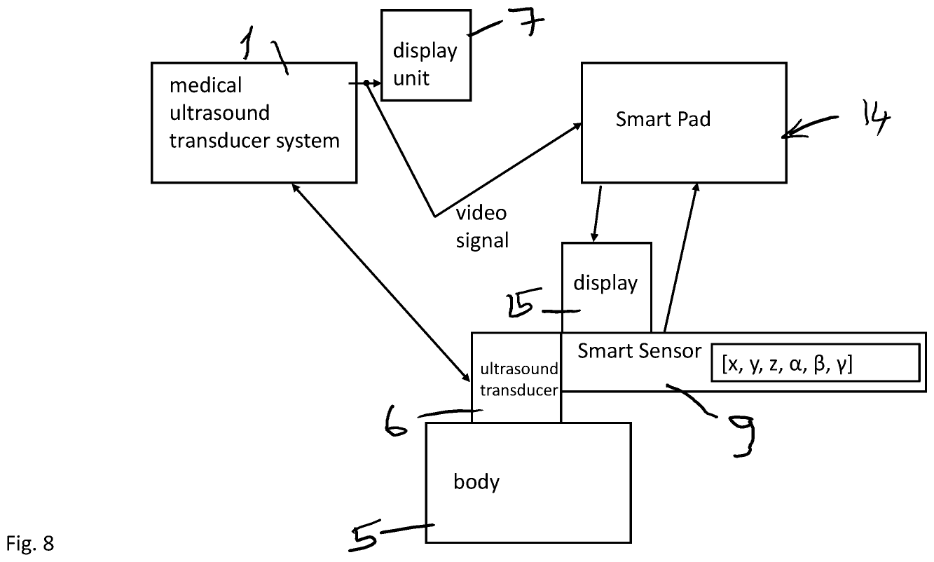

FIG. 8 shows a schematical representation of a system for creating a spatial graphical representation 4 of an organ of a body,

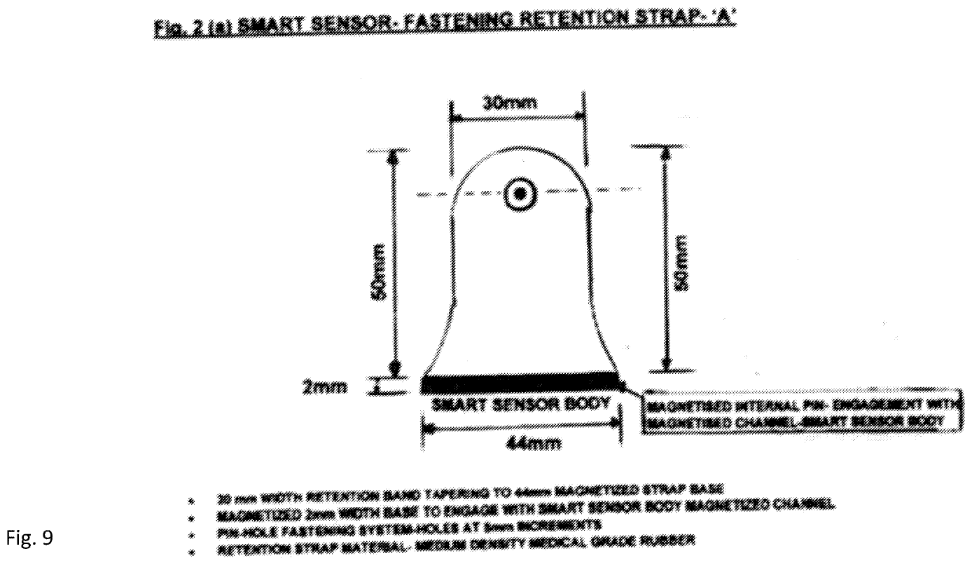

FIG. 9 shows a Smart Sensor 9 Fastening Strap,

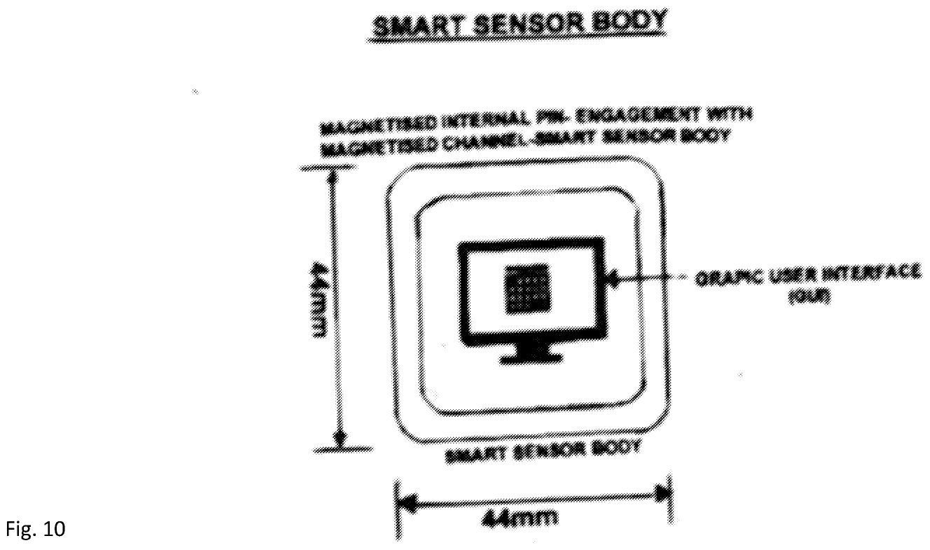

FIG. 10 shows a Smart Sensor 9 top view,

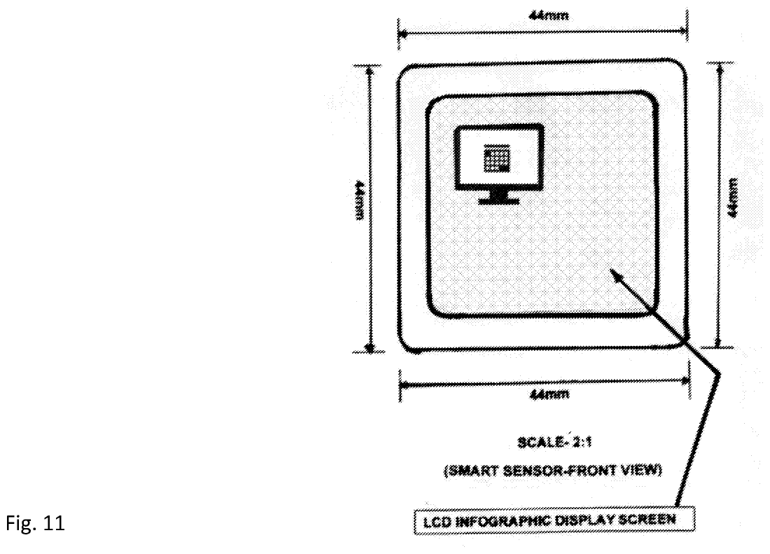

FIG. 11 shows a Smart Sensor 9 top view,

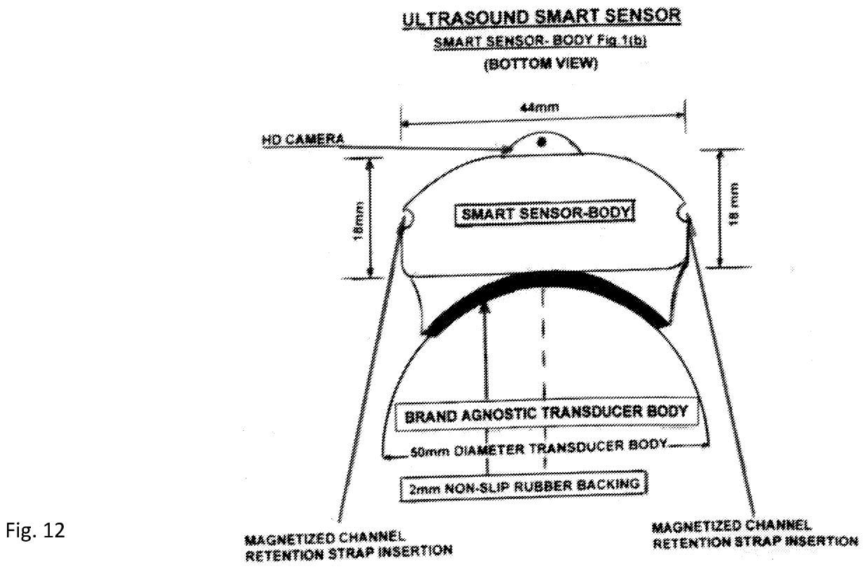

FIG. 12 shows a Smart Sensor 9 side view,

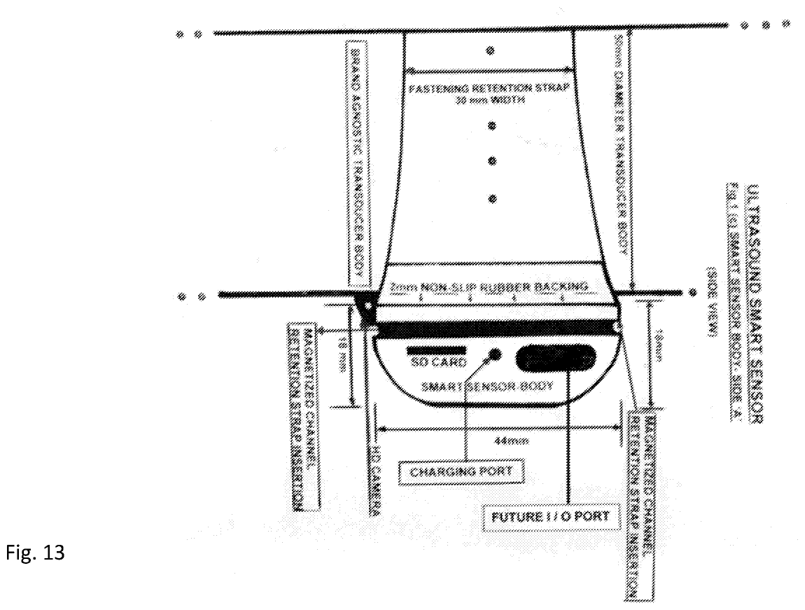

FIG. 13 shows a Smart Sensor 9 side view,

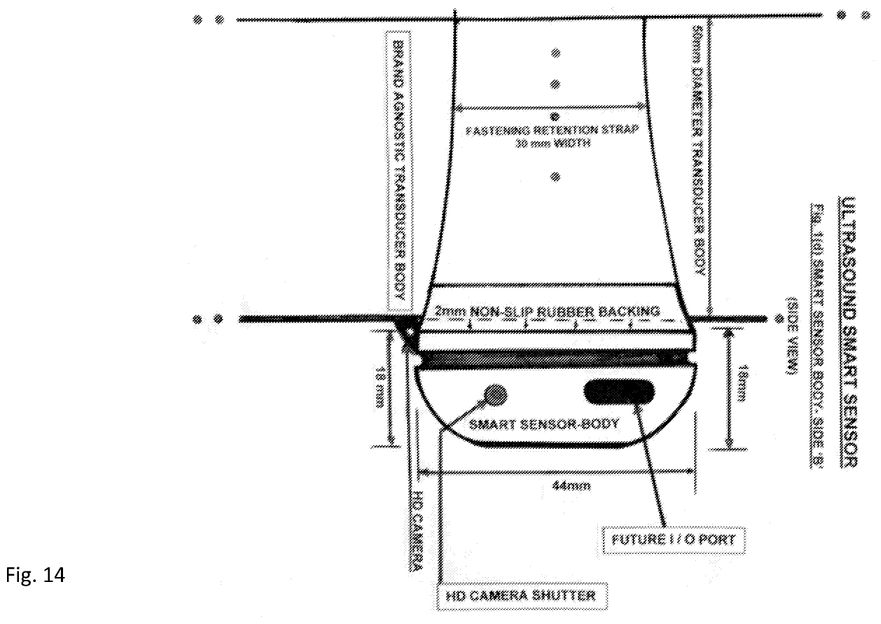

FIG. 14 shows a Smart Sensor 9 side view,

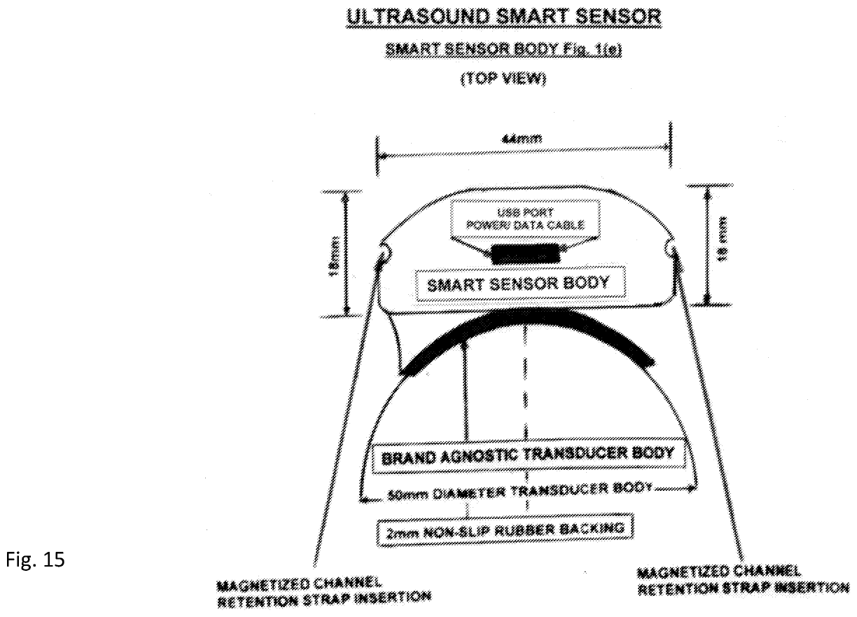

FIG. 15 shows a Smart Sensor 9 front view,

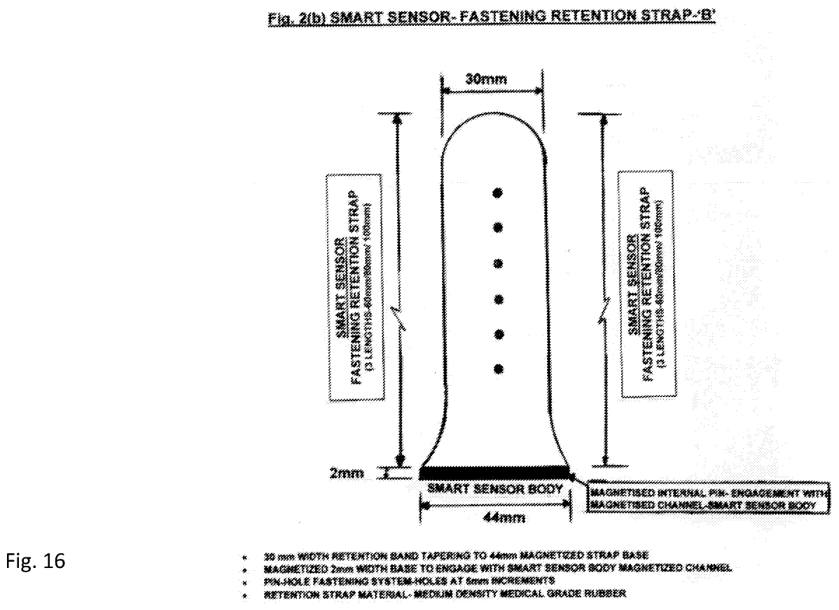

FIG. 16 shows a Smart Sensor 9 Fastening Strap,

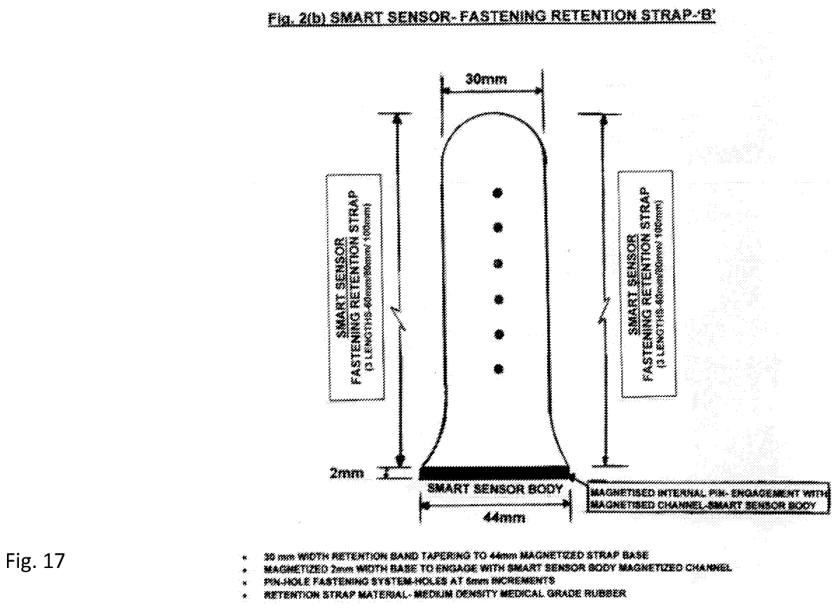

FIG. 17 shows a Smart Sensor 9 Fastening Strap,

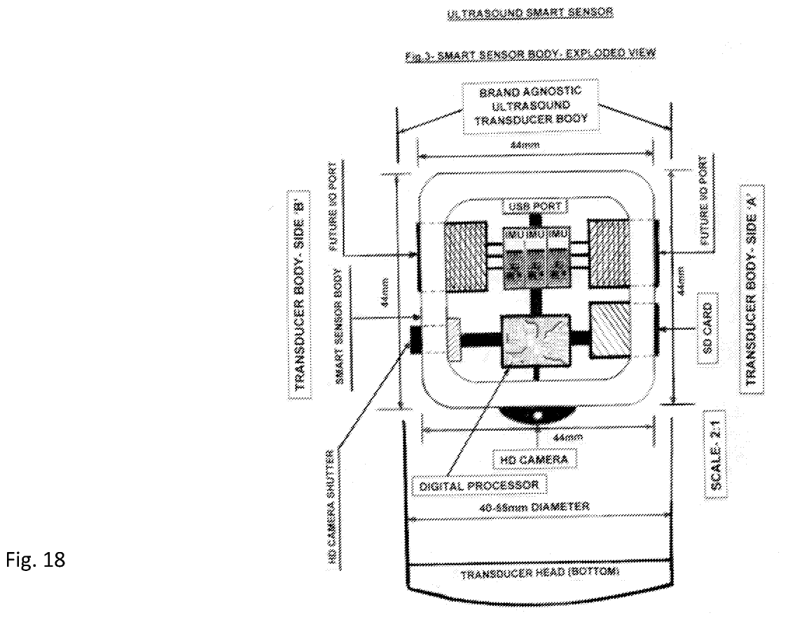

FIG. 18 shows a Smart Sensor 9 exploded view,

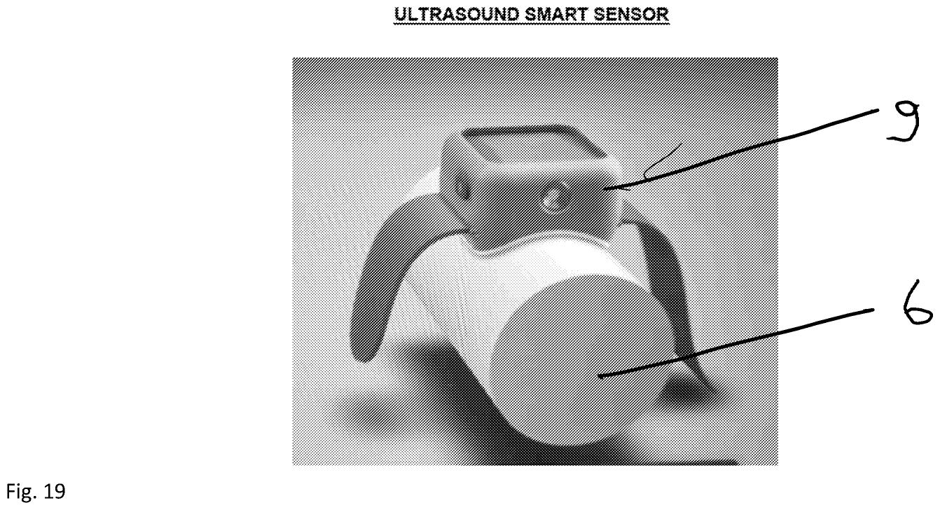

FIG. 19 shows a Smart Sensor 9 that is mounted to a transducer,

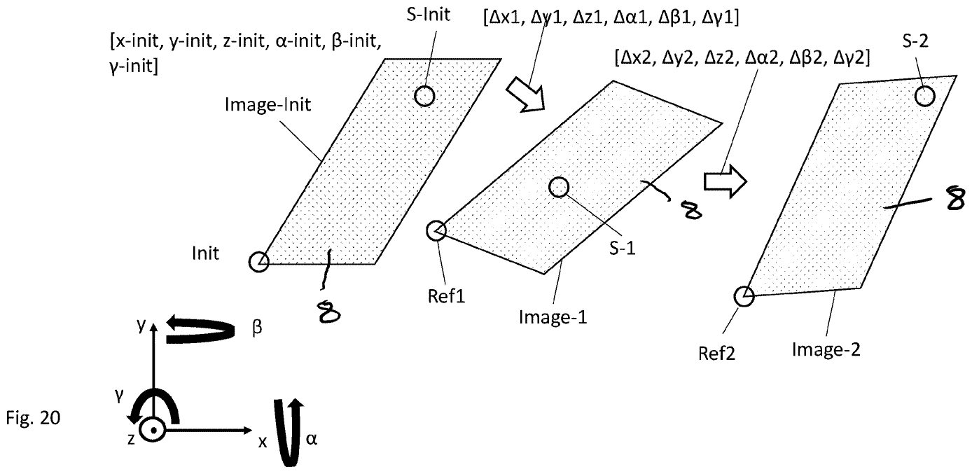

FIG. 20 shows three US images 8 with their respective attitude data.

FIG. 20 and FIG. 8 shows the method for creating a spatial graphical representation 4 of an organ of a body 5 from a multitude of 2-dimensional ultrasound images 8.

A medical ultrasound transducer system 1 with an ultrasound transducer unit 6 and with a display unit 7 for displaying a digital video signal as a 2D Ultrasound image 8 that represents what the ultrasound transducer unit 6 captures during the operation of the medical ultrasound transducer system 1 is shown in FIG. 8.

There is also a spatial attitude measurement unit 9 that is attached to the medical ultrasound transducer 11 unit, wherein the attitude measurement unit 9 comprises at least one linear accelerometer 10 for x, y, z and/or at least one rotational gyroscope 10 for each one of at least three spatial directions, α, β, γ, wherein the at least one linear accelerometer 10 and/or the at least one rotational gyroscope 10 are provided in MEMS technology.

A measurement computer system 14 is electrically connected with the display unit 7 and the attitude measurement unit 9, and that is provided for receiving and storing a plurality of digital video signals, and other common meanings of “signal” and a plurality of digital attitude measurement signals x, y, z, α, β, γfrom the attitude measurement unit 9.

A display unit 7—instead of integrating the Smart Sensor 9 Technology into the Medical Ultrasound transducer system 1—provides the video data.

The following is done in an initialization phase:
moving the medical ultrasound transducer 11 to an initial attitude [x-init, y-init, z-init, α-init, β-init, γ-init],
initializing the spatial position and/or orientation measurement unit [x, y, z, α, β, γ] and recording an initial video signal Image-Init together with an initial attitude [x-init, y-init, z-init, α-init, β-init, γ-init]

In a recording phase, the following is done:
moving the medical ultrasound transducer 11 to a series of consecutive delta attitudes [Δx1, Δy1, Δz1, Δα1, Δβ1, Δγ1], [Δx2, Δy2, Δz2, Δα2, Δβ2, Δγ2], a preceding delta attitude [Δx1, Δy1, Δz1, Δα1, Δβ1, Δγ1] being different from a following delta attitude, [Δx2, Δy2, Δz2, Δα2, Δβ2, Δγ2],
recording a series of delta video signals S-1, S-2 together with an associated series of delta attitude measurement signals [Δx1, Δy1, Δz1, Δα1, Δβ1, Δγ1], [Δx2, Δy2, Δz2, Δα2, Δβ2, Δγ2],

In an evaluation phase, the following is done:
inspecting the recorded initial video signal Image-Init and the recorded series of delta video signals Image-1, Image-2 for image points S-Init, S-1, S-2 that are characteristic of a surface of a pre-determined bodystructure, can be organs or other body 5 tissues 3, such as bones, tendons, muscles, ligaments, etc. and can also be a characteristic surface of a changed surface structure, such as a scar, or an interrupted or missing area, such as a wound,
determining the absolute positions of the characteristic surface image points S-Init, S-1, S-2 from the associated delta attitude measurement signals [Δx1, Δy1, Δz1, Δα1, Δβ1, Δγ1], [Δx2, Δy2, Δz2, Δα2, Δβ2, Δγ2],
providing a 3D-model of the pre-determined body structure by outputting the characteristic surface image points S-Init, S-1, S-2 together with their absolute positions. Their absolute position can be calculated by using their relative position in the video signals to a reference point Init, Ref1, Ref2, and by calculating the absolute position of these reference points as they move along with the series of delta video signals S-1, S-2. The orientation of the respective video signals/images has to be taken into account when calculating the respective absolute positions of the characteristic surface image points.

Whether of not a picture point is characteristic of a surface of a body structure is determined on the value of that picture point, which can be a color value, a brightness value, or other values of picture data. Pattern learning such as used in A.I. algorithms can be applied, too, when a decision has to be made on whether or not a picture point in one of the video signals is characteristic for a surface of a body structure.

FIG. 8 shows a schematical representation of a system for creating a spatial graphical representation 4 of an organ of a (living) body 5 from a multitude of 2-dimensional ultrasound images 8, with a medical ultrasound transducer system 1 with an ultrasound transducer unit 6 and with a display unit 7 for displaying a digital video signal as a 2D Ultrasound picture 8 that represents what the ultrasound transducer unit 6 captures during the operation of the medical ultrasound transducer system 1, and with a spatial attitude measurement unit 9 that is tightly attached to the medical ultrasound transducer 11, and with a measurement computer system 14 that is electrically connected with the display unit 7 and the attitude measurement unit 9, and that is provided for receiving and storing a plurality of digital video signals, wherein the term “signal” represents the signal line, the electric signal on that signal line, over time, also the individual digital data sets that represent the pictures that are displayed on the display unit 7, and other common meanings of “signal”.

Smart Sensor 9, can be integrated into a transducer head 6 or Stand-alone, attachable to any existing Ultrasound transducer head 6.

Three accelerometers 10, plus integrating twice for obtaining a displacement information.

Anatomy labelling, Voice-to-text: comments are stored at specific images 8.

HD Camera 111 for external anatomical reference.
Step #1: Reach defined target anatomy after being attached to the skin (Ground Zero as a reference point for subsequent measurements),
Step #2: move around as much as you can in order to capture whatever you can. Movements are measured with the acceleration transducers,
Step #3: use the number of captured image, together with the location and orientation data from the transducers for stitching the images 8 together.

Ultrasound has static images 8, but the 10 sec video with 30 frames/sec gives 300 images 8. That can be used to generate a 3D image from dynamic measurements (“images 8, 211, 212, and 213 are very good representation of that organ, let's use them”, overlap them, average them.

Start with a manual manipulation, and then use a robot.

Training purposes, correct online.

Smart Pad 14

Adds further functionality

Coordinates between the smart sensor 9 and a transducer head 6

Further features

Pressure, temperature, and microelectrical sensing (neurological) diagnostic capabilities

Goal: systems integration between smart sensor 9 and smart pad 14

The Smart Sensor 9 provides real time feedback on the 2D image capture process in of itself stand alone capability.

With the addition of a Smart Pad 14 provides both secondary imaging data which takes the 2D images 8 and provides the ‘Z’ axis and edge computing to stich the 2D images 8 into a 3D matrix.

Secondary capability of the Smart Pad 14 is with a nano liquid metal filament a medical diagnostic function for pressure and sensitivity (neurological) and temperature (circulatory—blood perfusion)

As the initial iteration is ‘brand agnostic’ it is more apt to prove the actual concept to approach a major portable US Manufacturer with the idea for them to internalise the functions and capabilities into their US Transducer head 6.

The initial iteration is externally ‘strapped’ t any version of a Transducer head 6 and subsequently calibrated (distance) from the transducer head 6 to skin contact—a standardised distance will be established in the first iteration with the goal again to have it adopted and internalised into the actual transducer head 6.

The Ultrasound images 8 from a Transducer have a variable field depth of imaging-as the anatomy rests upon the Smart Pad 14 the distance can be measured (there is digital calibration depth inherent in transducers) accurately between the Smart Sensor 9 and the Smart Pad 14 providing a reference depth . . . this is the Z coordinates.

Preferred Gyroscope Sensors 10 in the sense of the application are provided as Microelectromechanical systems (MEMS) and they include axial accelerometers and gyroscopes. These sensors are packaged as integrated circuits (ICs) and they are used are often used together, as accelerometers measure linear acceleration or directional movement, while gyroscopes measure angular velocity, tilt, or lateral orientation.

A gyroscope sensor 10 is a device capable of measuring and maintaining an object's orientation and angular velocity. These sensors are more advanced than accelerometers, as they can measure tilt and lateral orientation, whereas accelerometers can only measure linear motion. Gyroscope sensors 10 are also referred to as angular rate sensors or angular velocity sensors and are used in applications where it is challenging for humans to sense an object's orientation.

Angular velocity, expressed in degrees per second, represents the change in an object's rotational angle per unit of time. To achieve more robust and accurate motion sensing in consumer electronics, gyroscope sensors 10 are often combined with accelerometer sensors.

Gyroscope sensors 10 operate using the Coriolis force principle. To measure angular rate, the sensor's rotation rate is converted into an electrical signal. A common type of gyroscope sensor 10 is the vibration gyroscope, which consists of an internal vibrating element made of crystal material shaped like a double-T structure. This structure includes a stationary central part with sensing arms attached and drive arms on both sides.

The double-T structure is symmetrical, allowing for continuous lateral vibrations when an alternating electrical field is applied to the drive arms. As the drive arms are symmetrical, they cancel out leaking vibrations, keeping the stationary part and sensing arm static. When an external rotational force is applied, vertical vibrations occur in the drive arms, causing the stationary part to rotate and create vertical vibrations in the sensing arms. These vibrations are measured as a change in electrical charge, which is then used to measure the external rotational force applied to the sensor as angular rotation.

Gyroscope sensors 10 come in various sizes and performances, ranging from large devices like ring laser gyroscopes to small vibration gyroscopes. Based on their size, they can be divided into small and large-sized categories. Vibration gyroscopes are the most popular due to their small size and ease of use. Their accuracy depends on the stationary element material and structural differences, leading manufacturers to experiment with different materials and structures.

### Conversion of Standard Brand Agnostic Ultrasound Transducers 6 with a Smart Sensor

This application describes the conversion of Medical Device Industry Standard, Brand-Agnostic, Ultrasound Transducers used in Ultrasound Medical Imaging of the human anatomy. The Ultrasound Smart Sensor 9 will be a stand-alone Medical Device capable of attachment to the body 5 of any existing Ultrasound Transducer's body 5. The addition of 3-axis gyroscopic sensors, Inertial Measurement Units, as well as real-time monitoring and positional feedback and data recording capabilities will serve to optimize the Ultrasound Imaging capture process.

The objective of this Ultrasound Smart Sensor 9 Medical Device is to provide Sonographers with a real-time feedback mechanism of the Ultrasound Imaging capture process. This capability will result in the Standardization of Ultrasound Images 8 and improved Ultrasound image 8 and data quality. Standardized Ultrasound images 8 of optimized quality are required for the use of Artificial Intelligence and Machine 1 Learning on the imaging data to accurately stitch 2D to 3D Ultrasound images 8, and the subsequent development of Al driven Ultrasound Imaging Diagnostic software capabilities.

One objective is to convert Medical Industry standard, brand-agnostic Ultrasound Transducers with the addition of an attachable Smart Sensor 9. This Smart Sensor 9 will provide Sonographers with a real-time feedback mechanism regarding the Ultrasound Image 8 capture process as to the monitoring, tracking, positioning and Ultrasound Transducer coordinates in sub-millimeter precision. The conversion of Standard Ultrasound Transducers with the addition of an attachable Smart Sensor 9 will result in reproducible, consistent Standardized Ultrasound Images 8 and improved Ultrasound Image 8 quality of target organs, resulting in the following benefits:
Improved 2D Ultrasound image 8 quality
Provide a real-time feedback mechanism to Sonographers to optimize and quantify the Ultrasound image 8 capture process providing reproducible and consistent Standardized UltrasoundD images 8.
Provide a real-time feedback mechanism regarding the Ultrasound Transducer head 6 positioning, tracking and velocity in sub-millimeter accuracy during the image capture process whether by a Sonographer or by an Automated Robotic Image capture process to the primary Ultrasound parent device control panel and informatics through a USB data and power cable (FIG. 5) (FIG. 3).
The Smart Sensor 9 will have a SD Card Port (FIG. 6) (FIG. 3) for the storage and retrieval of all Smart Sensor 9 performance data.
Provide real-time data during the scanning process regarding the Ultrasound Transducer head 6 patterning, tracking and percentage coverage of the target organ on the Smart Sensor 9's LCD display screen (FIG. 1) (FIG. 3).
Increase Ultrasound Image 8 Diagnostic qualitative and qualitative features and real-time image capture performance data.
Enable Standardized 2D Ultrasound Images 8 to be accurately ‘stitched’ into 3D Ultrasound images 8 utilizing the sub-millimeter accurate coordinates recorded in real-time by the Smart Sensor 9 of the 2D Ultrasound images 8.
Enable more accurate Interpretation and Diagnostics resulting in faster treatment decisions.
Reduce misdiagnosis by utilizing Standardized Ultrasound Images 8 with enhanced image quality thus enabling accurate 2D to 3D stitching of the captured 2D images 8.
The Smart Sensor 9 has a HD Camera 111 (FIG. 4) (FIG. 8(d)(f)) and Shutter mechanism (FIG. 7) (FIG. 3(a)(f)) focused on the targeted anatomy being examined in real-time, providing complementary visual imaging data further corroborating the Ultrasound image 8 data for a more accurate assessment and subsequent Diagnosis of the target organs and soft tissues 3.
The Smart Sensor 9 shall have a KINETIC ENERGY HARVESTING capability to capture the energy produced by the Sonographers movements during the Ultrasound image 8
Provide a real-time feedback mechanism for the Academic Instruction of Ultrasound Image 8 capture processes, procedures, and protocol. PROCESS Standardized 2D Ultrasound images 8 are acquired utilizing the Smart Sensor 9 attached to any standard Brand-agnostic Ultrasound Transducer's body 5. The real-time data from the 3D-axis Gyros/Inertial Measurement Units (IMUs) provide the Sonographer or Automated Robotics a real-time feedback mechanism regarding the UltrasoundD image capture process.
The Smart Sensor 9 provides the mechanism for obtaining quantified real-time image capture data for repeatable, consistent image capture process, resulting in high quality, Standardized 2D Ultrasound images 8.
The Smart Sensor 9 provides the necessary Ultrasound image 8 capture process and real-time performance data to the Sonographer or an Automated Robotic Image Capture Device by employing the Smart Sensor 9's 3D-axis Gyro-Inertial Measurement Unit (IMU) Sensors.
The Smart Sensor 9 is securely fastened (FIG. 2(a)(b)) (FIG. 3(a)(b)(c)) to the base of the industry standard, Brand-agnostic Ultrasound Transducer, providing real-time data regarding the Transducer position relative to the targeted anatomy in sub-millimeter precision.
The real-time data of the Ultrasound Transducer's positioning, image capture patterning, tracking, velocity, and percentage of targeted anatomy imaged, is transmitted to the Smart Sensor 9 LCD Display (FIG. 1) (FIG. 8(e)(f)(g)), and via the UBS Data and Power cable (FIG. 5) (FIG. 8(b)(g)) to the Transducer's original base Control and Monitoring unit.
The Smart Sensor 9's also incorporates Kinetic Energy Harvesting hardware, capturing the energy from the Sonographer or Automated Robotic unit's movements.
The Smart Sensor 9's data is processed and the 2D imaging data is anatomically annotated and labelled in real-time, and the targeted anatomy's co-ordinates are displayed in sub-millimeter precision for every 2D Ultrasound image 8.
The Smart Sensor 9 has the capability to display the real-time scanning process, the sub-millimeter precise coordinates, and targeted anatomy's labelling, which is essential for real-time stitching for the Standardized 2D images 8 captured into 3D Ultrasound images 8.
The Smart Sensor 9's Programming software provides real-time feedback from the Smart Sensor 9 Ultrasound guiding the Sonographer or Automated Robotics for a Standardized Ultrasound Image 8 capture process.

### Method to Analyze 2D and 3D Ultrasound Images in Real-Timeabstract

This application describes the design and functions of a Grid based 3-axis Ultrasound SMART PAD with pressure, temperature, and micro-electrical sensing Medical Diagnostic capabilities. The Ultrasound SMART PAD also serves to track, monitor and record real-time data and coordinates of a SMART SENSOR in sub-millimeter precision of the image capture process for 2D Ultrasound images 8, and the conversion of those 2D Ultrasound images 8 into 3D Ultrasound images 8. One objective of this application is to provide a feedback mechanism to optimize and quantify the Ultrasound image 8 capture process which will enable the possibility of high quality, Standardized Ultrasound Images 8.

The Ultrasound SMART PAD can function as a ‘stand-alone’ Medical Diagnostic Instrument for Neurological assessments of Sensory and Motor functions as well as Vascular Assessments of the target organ. Another objective and capability is that the SMART PAD can also function in an Integrated Systems capacity with a Ultrasound SMART SENSOR. This SMART PAD and SMART SENSOR Integrated System will provide real-time feedback regarding the position, patterning, tracking and pressure of the Ultrasound Smart Sensor 9 during the 2D Ultrasound image 8 capture process. The SMART PAD-SMART SENSOR integrated system will also provide real-time data regarding the percentage of the identified target organ's total contact surface imaged during the Ultrasound imaging process.

This subject matter of the application will serve as a real-time and dynamic Ultrasound Imaging and Diagnostic Instrument to Medical Practitioners and Sonographers. The SMART PAD will capture and transmit in real-time the Sensory and Motor Neurological Diagnostic data to the Real-Time Data Processor (FIG. 7(d)) via a USB Data Cable. The SMART PAD will capture and transmit in real-time the Vascular Diagnostic's quantified data of the target organ's pulse and circulatory status to the Real Time Data Processor (FIG. 7(d) via the USB Data Cable. PROCESSSTAND ALONE CAPABILITIESThe ULTRASOUND SMART PAD as stand-alone Medical Diagnostic Instrumentation, will capture the contact surface of the hand, and through its quantitative assessment capabilities have the means to:
Quantify and map gross contact shape and surface area.
Measure quantitively the pulse of the Target Organ.—Measure the temperature at the contact surface area with the SMART PAD's Grid, providing a—Vascular assessment of the blood circulation and tissue perfusion status of the target organ.
Vary the temperature of the SMART PAD at designated Grid contact points as a Neurological assessment of the sensory status of the target organ.—Apply micro-electrical impulses at designated target organ contact points on the SMART PAD's Grid as a Neurological assessment of sensory status of the target organ.

### Smart Pad Integrated System with an Ultrasound Smart Sensor

Functioning in an integrated system with a SMART SENSOR, the SMART PAD will serve as the 3D Axis Grid Base for real-time monitoring of the SMART SENSOR, which is a standard, brand agnostic Ultrasound Transducer fitted with a 3D Motion Sensor. The SMART PAD will have a Power Supply and a USB compatible Data Port-Data Cable on the base of the SMART PAD (FIG. 4: ULTRASOUND SMART PADD Base Plate). The SMART PAD will be capable of mapping the target organ's contact shape and surface area, transmitting this data from the Ultrasound SMART PADD Embedded Grid Base Plate (FIG. 5 and FIG. 6(a)(b)(c)(d) inclusive), to a Real Time Data Processor (FIG. 7(c)) and subsequently to the Graphic User Interface (GUI)(FIG. 7(d) in real-time via the USB Data Cable. Providing the data and real-time feedback system on the Graphic User Interface display (FIG. 7(d) to a Sonographer/Medical Practitioner for the real-time monitoring of the position, patterning, tracking and the pressure of the SMART SENSOR's USB Data Cable connected to the Rea-Time Data Processor (FIG. 7(c).

### Impact

The capability to provide real-time data and a feedback mechanism regarding the entire Ultrasound Image 8 capture process to Sonographers will optimize 2D Ultrasound Images 8. Standardized 2D Ultrasound images 8 are currently lacking and is one of the primary deficits and obstacles in the wide-spread adoption of Ultrasound Imaging as an effective Diagnostic tool. Ultrasound Instrumentation has a quantum reduction in equipment capital costs for Institutions and Medical Practitioners in comparison to XRAY, MRI and CT-Scans. If Ultrasound image 8 quality can be brought up to at least MRI quality for soft-tissue imaging and diagnostics, the global medical impact will be profound. Ultrasound Images 8 may be captured in a Medical Specialist's office or in Military and Sport field locations and rural geographical locations. This will open up affordable, quality Medical imaging and Diagnostics to the vast majority of remote rural, un-serviced areas who currently do not have access to Primary Care Hospitals and Imaging Facilities.

High qualityD Ultrasound images 8 may be utilized on an essentially unlimited ‘Image Quota” basis, allowing the ongoing monitoring of a patient's progress and confirmation of their Medical Prognosis. This is an essential development for the ‘next-generation’ of Smart Medical Implants to assess on an ongoing basis their functional status and bio-integration.

Standardized Ultrasound Images 8 and the resultant significant improvements of Ultrasound Imaging qualitatively will result in reduced misdiagnosis, increased Intra/Inter Rater Diagnostic concurrence, and enable more rapid and accurate Treatment decisions by Medical Practitioners with improved Diagnostic imaging quality and the subsequent possibility of effective AI/Machine 1 Learning analytics and interpretation.

High quality Ultrasound Images 8 will provide significantly reduced dangers to patients as Ultrasound exposes patients to no lonizing Radiation as incurred with XRAY and CT-Scans, or the distress incurred during MRI imaging. Enhanced and optimized Ultrasound Image 8 quality is an absolutely necessity for accurate and effective 2D to 3D US Image ‘Stitching’ capabilities and a dramatic improvement in the resultant Ultrasound Image 8 quality. Standardized Ultrasound images 8 are mandatory for any subsequent AI Interpretation and Machine 1 Learning for the development of any AI assisted Ultrasound Image 8 Diagnostics capabilities and services.

Smart Pad 14 features are:

Washable—water and alcohol proof. Must be capable of standard medical-grade sterilization processes and subsequent reusability.

All materials utilized in the design and manufacturing process must be compliant with all global medical regulatory agencies and authorities for human skin contact.

Smart pad 14 contact materials with target organs-human skin must possess non-slip surface characteristics. Smart pad 14 grid material used in the 50 mm ‘target organ contact interphase (FIG. 2(b)(c)(d); FIG. 3(c); FIG. 4) material density and elastic deformity characteristics must be capable of capturing surface pressure data accurately in real-time via the embedded grid nano liquid metal-alloy eutectic filament FIG. 5; FIG. 6(b)(d).

The smart pad 14 grid must be capable of applying a micro-electrical current at specific designated smart pad 14 grid (FIG. 5; FIG. 6(b)(d)) coordinates as a neurological diagnostic tool.

The smart pad 14 base plate (FIG. 2(c); FIG. (3(c); FIG. 4; FIG. 5) capable of implantation of a liquid metal-alloy eutectic nano-filament (FIG. 5; FIG. 6(a)(b)(c)(d) in a precise square millimeter plus ‘cross ‘X’ grid pattern in a 2D (FIG. 5 ; FIG. 6(a)(b)(c)) and a 3D grid (FIG. 5; FIG. 6(d)) and possess materials of sufficient strength and zero deformity. Grid referenced ‘X’ axis-horizontal axis in a numerical configuration (FIG. 5; FIG. 6(b)). Grid referenced ‘Y’ axis-vertical axis in a numerical configuration (FIG. 5; FIG. 6(b)). Grid referenced ‘Z’ axis-base plate depth@5 mm for 3D grid referencing (FIG. 6(d)).

LED tricolor axis self-test mechanism on the real-time processor (FIG. 7(c)).

The smart pad 14 will supply all diagnostic and smart sensor 9 data generated via a USB cable systems integration graphic user interface (GUI) (FIG. 7(d)). Ultrasound smart pad 14-software capabilitiesReferencing the liquid metal-alloy eutectic nano-filament embedded square millimeter ‘cross ‘X’ dual diagonal grid (FIG. 5; FIG. 6(a)(b)(c)(d)) analytical capabilities as to the targeted gross anatomical shape capture and subsequent real-time imaging conversion via the real-time data processor and digital replication (FIG. 7(d)) and subsequently displayed on the graphic user interface (GUI) (FIG. 7(d)).

Analytical quantification of the anatomical 2D ultrasound imaged surface area coverage by the smart sensor 9 of the targeted anatomy. Analytical calculation of the measured pressure at each specified grid reference position (i.e., ‘X: 123-189: Y: 22-143: Z: 001-568’) quantified, indexed, and color-coded through the real-time data processor (FIG. 7(c)).

Systems integration and calibration between the smart sensor 9 data (transducer head 6 positioning/velocity/pressure mapping/optimized head patterning/percentage targeted anatomy surface area coverage) and the smart pad 14 (FIG. 1.0; FIG. 2; FIG. 3; FIG. 4; FIG. 5).

Closed-loop monitoring system—real-time data processor (FIG. 7(c)) for smart sensor 9 to an ultrasound smart pad 14 (FIG. 1; FIG. 2; FIG. 3(a)(b)(c)(d); Fig contact surface interphase monitoring and quantification.

### REFERENCE NUMERAL LIST

1 machine 1
1 medical ultrasound transducer system 1
3 tissues 3
4 spatial graphical representation 4
5 body 5
6 ultrasound transducer head 6
6 transducer head 6
6 STANDARD BRAND AGNOSTIC ULTRASOUND TRANSDUCERS 6
6 ultrasound transducer unit 6
7 display unit 7
8 ultrasound image 8
8 images 8
8 picture 8
9 smart sensor 9
9 spatial attitude measurement unit 9
9 attitude measurement unit 9
three accelerometers 10
gyroscope sensors 10
gyroscope sensor 10
at least one linear accelerometer 10
at least one rotational gyroscope 10
medical ultrasound transducer 11
pre-determined body
body structures 13
smart pad 14
measurement computer system 14
display screen unit 15
display input connector 16
attitude input connector 17
video input connector 18
digital attitude measurement signals 19
hd camera 111

1. A method for creating a spatial graphical representation of an organ of a body from a multitude of 2-dimensional ultrasound images 8, the method comprising the following steps:
providing a medical ultrasound transducer system with an ultrasound transducer unit and with a display unit for displaying a digital video signal as a 2D Ultrasound image that represents what the ultrasound transducer unit captures during the operation of the medical ultrasound transducer system,
providing a spatial attitude measurement unit that is attached to the medical ultrasound transducer unit, wherein the attitude measurement unit comprises at least one linear accelerometer and/or at least one rotational gyroscope for each one of at least three spatial directions (x, y, z, a, p, y), wherein the at least one linear accelerometer and/or the at least one rotational gyroscope are provided in MEMS technology,
providing a measurement computer system that is electrically connected with the display unit and the attitude measurement unit, and that is provided for receiving and storing:
a plurality of digital video signals and
a plurality of digital attitude measurement signals from the attitude measurement unit,
in an initialization phase:
moving the medical ultrasound transducer to an initial attitude, initializing the spatial position and/or orientation measurement unit, and recording an initial video signal together with an initial attitude in a recording phase:
moving the medical ultrasound transducer to a series of consecutive delta attitudes, a preceding delta attitude being different from a following delta attitude,
recording a series of delta video signals together with an associated series of delta attitude measurement signals,
in an evaluation phase:
inspecting the recorded initial video signal and the recorded series of delta video signals for image points that are characteristic of a surface of a pre-determined bodystructure,
determining the absolute positions of the characteristic surface image points from the associated delta attitude measurement signals,
providing a 3D-model of the pre-determined bodystructure by outputting the characteristic surface image points together with their absolute positions.

2. The method according to claim 1, wherein the recording phase comprises recording a series of digital camera signals together with the associated series of delta attitude measurement signals.

3. The method according to claim 1, comprising the step of reading digital information that is obtained from comparing the 3D-measurement model with at least one pre-recorded 3D-control model from a display and providing recording additional delta video signals.

4. The method according to claims 1, comprising the step of entering labeling data that is characteristic for body structures to recorded delta video signals.

5. A measurement computer system for providing a spatial graphical representation of an organ of a body from a multitude of 2-dimensional ultrasound images 8, the measurement computer system comprising:
a video input connector for receiving a digital video signal that a medical ultrasound transducer system sends to a display unit, the video signal comprising a 2D Ultrasound picture 8 that represents what the ultrasound transducer unit captures during the operation of the medical ultrasound transducer system, —an attitude input connector for receiving a digital attitude signal from a spatial attitude measurement unit that is attachable to a medical ultrasound transducer,
wherein the measurement computer system is provided for receiving and storing:
a plurality of digital video signals and
a plurality of digital attitude measurement signals,
wherein the measurement computer system is further provided for:
recording a series of delta video signals together with an associated series of delta attitude measurement signals, and for
inspecting the recorded initial video signal and the recorded series of delta initial video signals for image points that are characteristic of a surface of a pre-determined bodystructure, and for
determining the absolute positions of the characteristic surface image points S from the associated delta attitude measurement signals, and for
providing a 3D-measurement model of the pre-determined bodystructure by outputting the characteristic surface image points together with their absolute positions.

6. The measurement computer system according to claim 5, with a video input connector for receiving a digital camera signal from the spatial attitude measurement unit.

7. The measurement computer system according to claim 5, with a display output connector for sending digital information that is obtained from comparing the 3D-measurement model with at least one pre-recorded 3D-control model.

8. A spatial attitude measurement unit for providing a plurality of digital attitude measurement signals to a measurement computer system for providing a spatial graphical representation of an organ of a body from a multitude of 2-dimensional ultrasound images, the spatial attitude measurement unit comprising an attitude output connector for sending a digital attitude signal and a digital camera module for supplying a digital camera signal, wherein the spatial attitude measurement unit is attachable to a medical ultrasound transducer, wherein the attitude measurement unit comprises at least one linear accelerometer and/or at least one rotational gyroscope for each one of at least three spatial directions, wherein the at least one linear accelerometer and the at least one rotational gyroscope are provided in MEMS technology.

9. The spatial attitude measurement unit of claim 8, comprising a display input connector and a display screen unit for displaying digital information that is received on the display input connector.
